(19) United States
(12) Patent Application Publication
(10) Pub. No.: US 2012/0003293 A1
(43) Pub. Date: Jan. 5, 2012

(54) NITRIC OXIDE-RELEASING DRESSINGS

(76) Inventors: Chris Miller, North Vancouver (CA); Bruce Murray, North Vancouver (CA)

(21) Appl. No.: 13/126,931

(22) PCT Filed: Oct. 28, 2009
(86) PCT No.: PCT/CA2009/001563

§ 371 Date: Sep. 20, 2011

Related U.S. Application Data
(60) Provisional application No. 61/109,652, filed on Oct. 30, 2008.

(51) Int. Cl. A61K 33/00; A61L 15/44; A43B 13/38; A61F 13/00; A61M 35/00; A43B 17/00; A61L 15/18; A61P 31/00

(52) U.S. Cl. 424/445; 424/718; 602/48; 604/23; 2/239; 36/43

(57) ABSTRACT

A wound dressing comprising nitric oxide gas producing component.

### CROSS REFERENCE TO RELATED APPLICATIONS

This application is entitled to the benefit of and incorporates by reference essential subject matter disclosed in U.S. Provisional Patent Application No. 61/109,652 filed on Oct. 30, 2008 and International Patent Application No. PCT/CA2009/001563 filed on Oct. 28, 2009.

### FIELD OF THE INVENTION

This invention relates to the use of nitric oxide-releasing anti-septic agents in wound dressings. More specifically, this invention relates to wound dressings comprising compounds that release or generate nitric oxide which has an anti-sceptic effect.

### BACKGROUND OF THE INVENTION

Wound dressings and bandages are prevalent in wound care management. These dressings and bandages are useful in providing protective barriers for surface or subsurface lesions. Furthermore, wound dressings can absorb and draw off blood, serum or pus from the lesion to provide clean wound-sites which are conducive to healing. Dressings also promote healing by controlling and restricting water-loss, thus providing a moist environment that is favourable for healing. However, there are risks associated with wound dressings. For example, the wound dressing or bandage can raise the risk of wounds being infected or re-infected with pathogens, including bacteria, viruses, fungi and parasites. The exudation of serum and blood from wounds to the external environment and the difficulty in maintaining a sterile site, can lead to infection or re-infection of lesions because this rich medium of serum and blood, when trapped in a moist wound dressing, provides a repository of microbes and an opportune site for microbial growth.

It has been suggested that anti-microbial materials may be used in conjunction with a dressing in order to reduce the risk of infection or re-infection of a wound site. For example, some documents suggest using biocompatible anti-microbial metal ions such as silver, gold, platinum, palladium, iron, tin, copper, antimony, bismuth or zinc. Such anti-microbial metals have been incorporated into and onto surgical, wound and medical device dressings, bandages, and bio-absorbable materials, such as sutures, staples, prosthetic devices, microcapsules and the like, to avoid, prevent and treat bacterial, fungal and microbial infections. The use of free elemental iodine has also been suggested.

In addition to wound dressings and bandages for protecting and treating wounds, other methods of administering topical or systemic therapies to patients have been used to treat wound infections. For example, antibiotics have been used to treat infected abscesses, lesions, wounds, and similar injuries. However, an increasing number of pathogens have developed resistance to such therapy and some patients are allergic to the compositions used for treatment. Furthermore, it is known that infectious agents can interfere with the circulation of blood within an infected region. Such reductions in blood flow may lower the level of anti-septic agent that can be systemically delivered to the infected region. Topical applications of anti-septic agents can solve this problem but such methods often do not allow the anti-septic agent to penetrate in sufficient concentrations to be effective.

Nitric oxide (NO) is produced in the endothelium tissue of the human body as part of normal physiological processes. NO is an endogenous vasodilator i.e., an agent that widens the internal diameter of blood vessels. NO has also been investigated for its use as a sterilizing agent. It has been discovered that NO will interfere with or kill the growth of bacteria grown in vitro. WO00/30659 discloses a method and apparatus for the treatment of respiratory infections by NO inhalation. It has also been suggested that NO has an inhibitory effect on the life cycle of the influenza virus. See, for example, Rimmelzwaan et. al., Journal of Virology; Vol. 73, No. 10; p. 8880-8883 (October 1999) and Akerstrom et. al., Journal of Virology; Vol. 79, No. 3; p. 1966-1969 (February 2005). In addition, it has been suggested that NO gas may be delivered via a device in order to treat surface or subsurface wounds (U.S. Pat. No. 7,520,866; U.S. Pat. No. 6,432,077; U.S. Pat. No. 6,793,644; U.S. Pat. No. 7,122,018; J. B. J. Hardwick et. al., Clinical Science (2001) 100, 395-400).

### SUMMARY OF THE INVENTION

The present invention provides methods of reducing and/or eliminating microbes and spores within dressings. In certain embodiments, the present invention is aimed at reducing and/or eliminating the potential for re-infection of surface or subsurface lesions by pathogens, including those infections caused by viruses, fungi, parasites and bacteria, and those caused by pathogens that have developed resistance to one or more antibiotics.

The present invention further provides a wound dressing comprising a nitric oxide gas producing component. For example, the present wound dressings may comprise discrete supplies of sodium nitrite, citric acid, an oxygen-releasing compound, and an aqueous component. The nitric oxide of the present invention provides an anti-sceptic or sterilizing effect. The nitric oxide producing component is integral to the dressing making it convenient and easy to apply while the nitric oxide is delivered directly to the appropriate place.

As used herein, the term “dressing” means material used to cover a wound. Examples, of dressings include gauzes, tulle, semi-permeable films, hydrocolloid, polyurethane or silicone foams, hydrofibres, and the like. Combinations of dressings may be used herein.

As used herein, the phrase “nitric oxide gas producing component” means a constituent part of the dressing or apparatus is capable of discharging or generating nitric oxide gas.

According to an embodiment, the wound dressing comprises a layer comprising discrete supplies of sodium nitrite and citric acid and a separate layer comprising an oxygen-releasing compound layer. The dressing may comprise a supply of an aqueous gel and/or water embedded within a layer distinct from both the sodium nitrite-citric acid layer and the oxygen-releasing compound layer.

According to certain embodiments, the aqueous component is provided as hydro-gel capsules comprising the aqueous component or breakable packets comprising the aqueous component.

According to certain embodiments, the water is administered by the user or another person. The water may be absorbed (i.e., wicked) by the dressing and/or provided in frangible packets comprising of sterile water.

The present invention provides, wound dressing comprising a manipulably controllable device which releases a supply of aqueous gel or water upon activation. The activation may, for example, be triggered by the user or by a care giver. The aqueous gel or water may, for example, come from hydro-gel capsules or breakable packets. Post-release the aqueous gel or water will mingle with the sodium nitrite, citric acid, and oxygen-releasing compound to produce NO.

The present invention also provides an apparatus configured for use in the proximity of a subject's foot or within a subject's footwear and configured for deployment of anti-microbial gases. An example of the present apparatus is an inner-sole to be placed within a subject's footwear. The apparatus may be a sock to be worn on a subject's foot comprising NO-producing agent. The present apparatus may comprise discrete supplies of sodium nitrite, citric acid, an oxygen-releasing compound, and an aqueous gel or water, similar to the composition of the wound dressing described above.

The present dressings and apparatus preferably comprise supplies of sodium nitrite and citric acid that would result in a concentration of gaseous NO of from about 8,000 to about 12,000 ppm. It is desirable that the NO persists from about 45 minutes to about 90 minutes.

The present dressings and apparatus preferably comprise supplies of an oxygen-releasing compound embedded in a layer such that the resultant concentration of gaseous oxygen is greater than 18%.

The present dressings and apparatus may comprise a bottom layer that is permeable to gaseous NO. For example, the bottom layer may be a fibrous or foam material. The bottom layer may be separated from any other layers by a membrane that is impermeable to larger molecules such as nitrogen dioxide but permits the diffusion of NO.

The present dressings and apparatus may comprise an upper surface barrier that is relatively gas and moisture-impermeable to prevent premature dispersion of gaseous NO. The barrier may also prevent the premature activation of the NO producing reaction by water molecules in the surrounding environment.

The present invention also provides a method of providing anti-septic treatment to a subject using the present dressings and/or apparatus. The method may comprise the following steps:
(i) providing a dressing or apparatus configured for deployment in communication with at least a portion of a subject; the dressing or apparatus comprising a plurality of layers said layers comprising discrete supplies of sodium nitrite, citric acid, an oxygen-releasing compound, and an aqueous gel or water; and
(ii) controllably commingling said discrete components to release gaseous NO such that said gaseous NO communicates with the subject's surface.

The present dressings or apparatus may be used for treating prolerating microbes causing objectionable odours. A suitable method for such treatment is exemplified by the following steps: (i) providing an inner-sole or sock configured for deployment about a portion of a subject's foot wound; (ii) providing a plurality of layers in said inner-sole or sock; (iii) providing four discrete components in said layers of the inner-sole or sock; (iv) controllably commingling said discrete components to release gaseous NO; and (v) controllably diffusing said gaseous NO into the layer of the inner-sole or sock in contact with the subject's skin.

The present invention may be used in conjunction with currently available dressings. For example, the present invention may be used as an adjuvant to dressings comprising silver as their anti-microbial agent.

In some embodiments of the present invention it will be necessary for the NO gas to contact the subject directly in order to have the desired anti-microbial or sterilizing effect but the invention also encompasses embodiments where the anti-microbial effect is mostly confined to the dressing or apparatus itself.

### BRIEF DESCRIPTION OF THE DRAWINGS

FIG. 1 is a cross-sectional view of a wound dressing or bandage comprising a NO- and oxygen-generating anti-septic or sterilizing agent for reducing or eliminating microbial levels and spores within wound dressings and bandages. The dressing comprises an upper gas-impermeable & water-impermeable barrier membrane (10); a bottom gas permeable barrier membrane (12); a layer comprising NO-generating chemicals (14); a layer comprising aqueous gel (16); an activator device (18); and dressing filler material (20).

FIG. 2 is a cross-sectional view of an inner-sole in a shoe (40) comprising a NO- and oxygen-generating anti-septic or sterilizing agent. The apparatus comprises an upper gas-impermeable & water-impermeable barrier membrane (42); a bottom gas permeable barrier membrane (44); a layer comprising NO-generating chemicals (46); a layer comprising aqueous gel (48); an activator device (50); and dressing filler material (52).

FIG. 3 is a top view of a specialized six-well culture plate designed to conduct in vitro studies for evaluating the effects of NO— and oxygen gas-generating compounds on microbial cells. The setup allows various mixtures of compounds to be placed in the bottom of the culture plate. Further, there is a porous screen suspended just above the gas-generating material to accommodate a 1.5 cm² dressing sample. The dressing samples can be inoculated with inoculums containing various vegetative or spore forming microbes in various concentrations.

FIG. 4 is a top view of the specialized culture plate shown in FIG. 3 with an active compound generating gaseous NO below the porous screen wherein the inoculated test dressing resides.

FIG. 5 is a graphical representation of experimentation data showing the duration and concentration of NO, nitrogen dioxide and oxygen (not shown) that resulted from a specific NO-generating mixture.

FIG. 6 is an illustration of an experiment where various commercial dressings are being tested to ascertain optimal minimum inhibitory concentrations of NO gas generated from mixtures to eradicate bacterial loads of 1×10⁵ colony forming units per millilitre. These types of experiments may be used to confirm the additional adjuvant effect of NO-generating compounds with other anti-septic dressings.

FIG. 7 is a graphical representation of experimental data showing the complete elimination of a 5 log₁₀ cfu/mL of Staphylococcus epidermidis from a Staphylococcus epidermidis-infected dressing (Proguide, Smith & Nephew, United Kingdom).

FIG. 8 is a graphical representation of experimental data showing the added anti-microbial/sterilizing effect of a NO-generating compound on the anti-microbial effect of a commercially available silver foam dressing infected with Staphylococcus aureus.

### DETAILED DESCRIPTION OF THE INVENTION

The present invention relates to the use of nitric oxide (NO) as the anti-septic or sterilizing agent. NO appears to be a broad spectrum anti-microbial agent and can have a deleterious effect on many pathogenic organisms such as bacteria, viruses, fungi, parasites, etc. NO seems to cross cell membranes and is able to target a variety of marcromolecules. Additionally, since NO is endogenous to humans the potential for allergic reaction is much reduced. Furthermore, NO is known to be an endogenous vasodilator, able to widen the internal diameter of blood vessels, thus exposing wounds to NO may counter-act any constriction of the blood vessels caused by infectious agents.

The concentration at which NO is cytotoxic to microbes is generally lower than the level at which it is cytotoxic to mammalian cells. However, NO can be toxic to humans if high concentrations, especially concentrations greater than 1,000 ppm, are inhaled and enter the bloodstream. Even at lower concentrations of inhaled NO, gaseous NO can be harmful if the time of exposure is relatively high. Thus, it is preferred that any apparatus for treating infections with NO prevents or minimizes the risk of exposure of the subject to toxic concentrations of NO.

The present invention relates to assemblies, apparatus, and methods for treating and preventing various infections in surface or subsurface wounds or lesions, including those caused by viruses, fungi, parasites and bacteria, and those caused by pathogens that have developed resistance to one or more antibiotics.

The present invention provides for the delivery of NO gas into a wound dressing at effective concentrations for a relatively short period of time. This concentrates the anti-septic agent at the infection site and reduces or eradicates microbial burden within the wound dressing to avoid infection or re-infection of the wound.

The present invention may be used, for example, to treat chronic non-healing wounds, acute wounds, MRSA infections, and the like.

An embodiment of the present invention relates to wound dressings and bandages used for wound care management. In addition to providing a protective barrier and keeping a moist wound environment, dressings absorb fluids, remove exudates, pus and debris. However, because of this feature, a dressing can be a repository of microbes, which can then re-infect the wound. It has been shown that NO gas reduces the bacterial burden about 5-6 log₁₀ cfu/mL. In addition, NO can have an anti-viral and fungicidal effect. Moreover, NO gas can eradicate microbes in both their vegetative and spore phase. NO gas can be used in conjunction with dressings or as an adjuvant to commercially available anti-microbial dressings.

An embodiment of the present invention relates to assemblies, apparatus, and methods for reducing the pathogenicity of transmissible agents that are in or on a wound dressing, wherein the method comprises applying to a wound a dressing or bandage with a source of gaseous NO and releasing said NO into the dressing or bandage. Such dressings and methods will also be effective for treating wounds that are infected by pathogens. If the wound is infected by, the presence of the gaseous NO in the dressing will kill or otherwise reduce the pathogencity of infectious agents such as bacteria, viruses, fungi, protozoans, or other pathogens.

The present dressings comprise a source of gaseous NO. The source may comprise sodium nitrite and citric acid. The reaction between sodium nitric and citric acid may be catalyzed by the presence of an aqueous gel or water. Accordingly, the present invention preferably comprises a source for an aqueous gel or water. The source may comprise hydro-gel capsules or packets comprising an aqueous gel. The hydro-gel capsules or packets may be configured so as to release the aqueous component upon the controllable activation of a device by the user. A suitable device for activating the aqueous component may comprise a pull tab attached to the hydro-gel capsules, which when pulled, breaks the capsules to release the inner aqueous component. Such device may also comprise aqueous gel or water packets, such packets being broken to release the inner aqueous component by applying pressure over the wound dressing. The commingling of the sodium nitrite, citric acid, and aqueous component produces gaseous NO which can diffuse through at least a portion of the wound dressing. Application of the wound dressing or bandage over a surface or subsurface lesion may also result in subsequent absorption of liquid into the dressing from the wound, which could act as a further catalyst for the NO-producing reaction.

An embodiment of the present invention relates to assemblies comprising an upper surface layer and a bottom surface layer. The upper surface layer comprises a moisture- and/or gas-impermeable barrier such as a membrane, wherein the barrier reduces or prevents the dissipation of the NO into the atmosphere. The barrier may also reduce the risk of premature catalyzation of the reaction between the sodium nitrite and citric acid by preventing environmental moisture from penetrating to catalyze the NO-producing reaction. The bottom surface layer of the wound dressing preferably comprises a gas-permeable membrane to allow the dispersion of the NO gas through the bottom surface barrier of the wound dressing. It is within the scope of the present invention to also have the bottom layer of the dressing comprising a fibrous or foam material for contact with the subject's skin. Such fibrous or foam material may be separated from the upper layers by a membrane that is permeable to small gas molecules such as NO, thereby only allowing the gaseous NO to diffuse into the fibrous or foam material. Preferably the membrane is impermeable to large molecules, such as nitrogen dioxide.

The half-life of NO is short, thus resulting in a short, localized effect of NO in treating a specific infection site. Wound care studies in humans have shown that the delivery of NO to an infection site does not have significant side-effects. Further, human cells are able to tolerate relatively high concentrations of NO. Therefore, with the correct parameters for delivering an effective dose in a short period of time, gaseous NO will be an effective therapy for treating surface or subsurface wounds and for reducing or eliminating the microbial burden within a wound dressing. Even if the effective NO concentrations are high, the skin cells adjacent to the wound should be able to cope and NO overspill from the dressing should not have a deleterious effect on the wound bed and its associated cell lines.

The sodium nitrite-citric acid components and the aqueous component of the present dressings are preferably separated such that production of the gaseous NO only occurs once the dressing is deployed. The quantity of sodium nitrite and citric acid embedded in the dressing is preferably at a level wherein a reaction between the sodium nitrite and citric acid will result in a concentration of gaseous NO capable of achieving its anti-septic or sterile effect within approximately 3 minutes to 1½ hours. For example, such concentrations may be about 5,000 to about 22,000 ppm.

Preferably the sodium nitrite and citric acid is contained in an upper layer of the wound dressing as compared to the aqueous component. The aqueous component may be contained within a layer immediately beneath the sodium nitrite-citric acid. Such aqueous component may comprise hydro-gel capsules or some type of similar capsule or packet that contains an aqueous component, said aqueous component released only upon controllable activation by the user. It is within the scope of the present invention to provide a controllable device configured to trigger the release of the aqueous component from its capsule or packet, and thereby catalyze the reaction between the sodium nitrite and citric acid to produce gaseous NO at such time when the user controllably activates the device.

Prior art devices for delivering NO to treat wounds have required a closed environment. However, it has been surprisingly discovered that the anti-microbial efficiency of NO is increased in the presence of oxygen. The oxygen may be environmental oxygen or may be delivered via an oxygen generating component within the dressing or apparatus.

The effect of various oxygen concentration levels on the efficacy of 1000 ppm treatment of NO was studied. The results are summarized in Table 1.

TABLE 1
Effect of varying oxygen concentrations
Compilation of 1000 ppm Exposures with
varying Concentration of oxygen

| | | CFU/mL | CFU/mL | Cfu/mL | Cfy/mL |
|---|---|---|---|---|---|
| | | 1 hour | 2 hour | 3 hour | 4 hour |
| Control | 1,000,000 | | 1,000,000 | | 1,000,000 |
| 0% Oxygen | | | 100,000 | | 100,000 |
| 14.5% Oxygen | | | 2730 | 78 | 0 |
| 21% Oxygen | | 100,000 | 10,000 | 0 | 0 |

Preferably the resulting gaseous oxygen concentration is greater than 18%. The NO may be delivered in the presence of an oxygen supplement. Any oxygen producing component may be used herein e.g. Aluminium oxide. The combination seems to provide an enhanced anti-septic and/or sterilization effect over the delivery of NO without an oxygen supplement. Thus, the present invention embodies this improved method of treating vegetative and spore forming bacterial, viral, protozoan and fungal infections by generating gaseous NO in the presence of oxygen. An oxygen-releasing compound may be contained in a separate layer from the sodium nitrite-citric acid layer and the aqueous component layer. Such oxygen-releasing layer may, for example, be located between the aqueous component layer and the bottom layer comprising the fibrous or foam material. The oxygen-releasing compound may be embedded into such layer of the wound dressing at a level wherein the concentration of oxygen produced is at a concentration greater than 18%.

The present invention includes dressings comprising sodium nitrite, citric acid, aqueous component, and oxygen-releasing component wherein the release of the aqueous component can cause the commingling of the sodium nitrite, citric acid, aqueous component, and oxygen-releasing component therein, resulting in the production of a mixture of NO and oxygen. The quantity of sodium nitrite-citric acid is preferably controlled to only allow the production of a certain concentration of gaseous NO, such quantity dissipating within approximately 3 minutes and 1½ hours, thereby applying a high concentration of NO over a short period of time within the dressing and/or onto the wound.

Another embodiment of the present invention relates to assemblies, apparatus and methods for reducing the pathogenicity of transmissible agents that have infected or could infect a wound on a subject's foot, wherein the subject is provided with an inner-sole or sock comprising a source of gaseous NO. The NO may be released into the inner-sole or sock so that it contacts the wound. Suitable sources for gaseous NO are described above.

The present inner-soles or socks may also be used in order to reduce or kill microbes or fungi which may be causing foot odour or other minor infections such as athlete's foot.

The NO-gas releasing mixture herein may comprise a discrete amount of potassium nitrate and/or a discrete amount of chromium oxide. Such a mixture is preferably provided in combination with a component configured to maintain a temperature within and about the dressing in a range from ambient to cooler than ambient.

The aqueous component herein may comprise one or more alternative supplies of water molecules, e.g., in frangible packets containing sterile distilled water. Alternatively, the NO-gas producing reaction may be catalyzed by water molecules from the subject's body fluids. In this embodiment it is preferred that the dressings or apparatus comprise portions configured for contacting such water molecules. Accordingly, it is possible to configure the wound dressings/apparatus of the present inventions to release of NO gas for the duration of time that the dressing is deployed on and about a portion of a subject's body surface.

The present invention is described with reference to specific details, preferences, and examples of particular embodiments thereof. It is not intended that such details and examples be regarded as limitations upon the scope of the invention except insofar as and to the extent that they are included in the accompanying claims. As used in this specification and the appended claims, the singular forms “a,” “an,” and “the” include plural referents unless the content clearly dictates otherwise. Unless otherwise specified all documents referred to herein are incorporated by reference.

### EXAMPLES

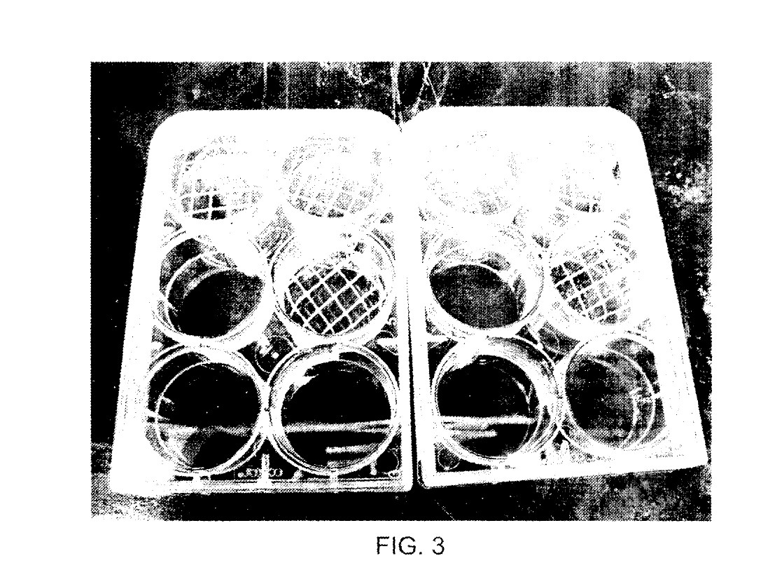

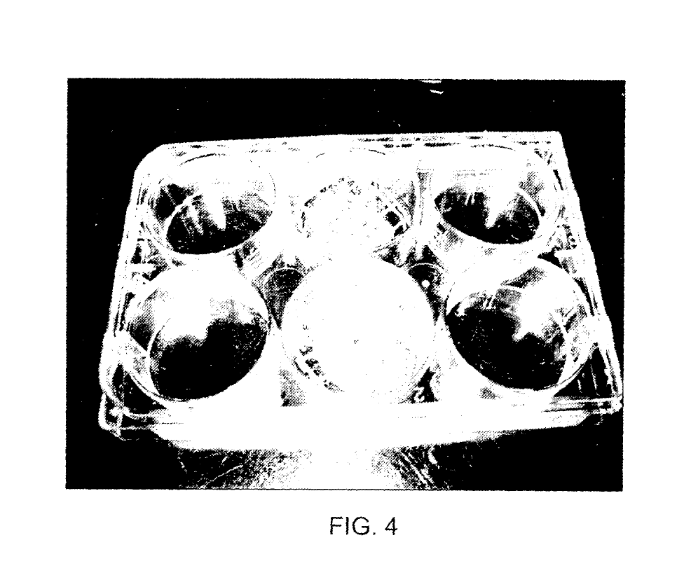

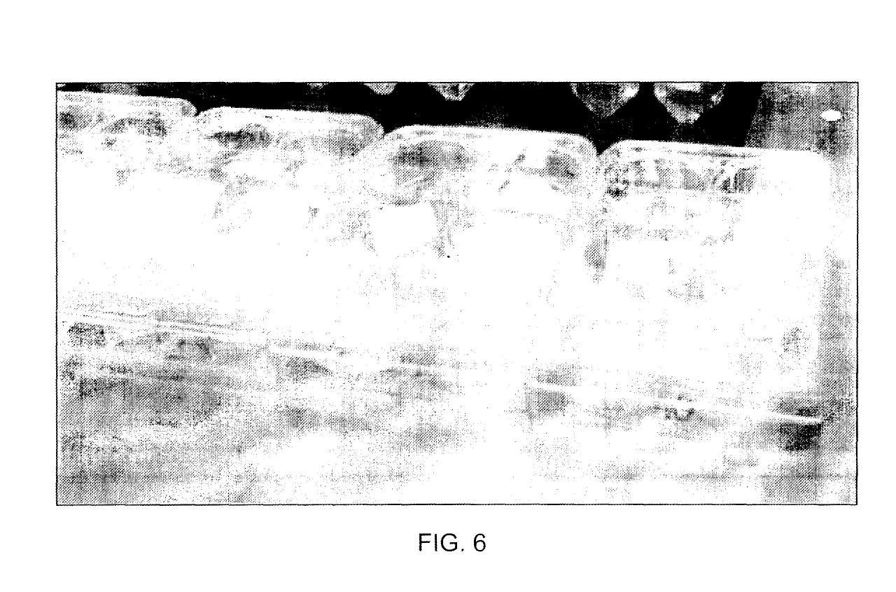

To study the potential effectiveness of NO-generating compounds on the bacterial load within a dressing, a custom exposure chamber was designed. A standard six-well culture plate (Corning 3516, Corning, N.Y.) was used. Standard Chicken Wire Mesh was cut to 3.5×3.5 cm squares, folded into a dome-like structure to be placed into the well. FIG. 3 shows how the mesh is inserted into the six-well plate in order to provide a separation between the reactive NO-generating mixtures in the bottom of the well from the dressing in the top of the well. Experiments were performed within a biosafety fume hood. Using a clean stainless steel spatula, active ingredients were weighed and placed into the bottom of each well and mixed thoroughly, as shown in FIG. 4. The Wire Mesh insert was placed into the well and then a 1.5 cm² bacterial-laden dressing (see below) was placed on top of the Wire Mesh (FIG. 6).

Bacterial-laden dressing inoculums were prepared using an American Type Culture Collection (ATCC) strain (14990) of Staphylococcus epidermidis. The organisms were grown according to the standard operating procedures of the microbiology laboratory. From these cultures, a 0.5 McFarland standard with 10⁸ cfu/mL was prepared and further diluted 1:1000 with sterile saline to 10⁵ cfu/mL in a volume of 20 mL. The concentration of 10⁵ cfu/mL was chosen as it is an accepted threshold for determining wound infection. Bacteria were suspended in 0.9% saline rather than nutrient broth media, because saline maintains the bacteria in a more representative in vivo state and in a more resistant state in which they neither multiply nor die. Further, suspensions in saline were standardized based on similar in vitro NO studies that have shown that substances found in a bacterial laboratory support media bind NO, and that this interference may have masked the true effects of NO in previous studies. Aliquots of 0.5 mL were then pipetted onto 1.5 cm² dressing samples, which were then placed on the Wire Mesh after which the six-well plate lid was put in place. Two wells in each plate were prepared for each time point during the studies and all studies were repeated three times.

At each time point sterile tweezers were used to remove the dressing and placed into a sterile 50 mL test tube containing sterile saline. The test tube was mixed vigorously for 20 seconds on an electric vortex machine to remove bacteria from the dressing. Samples of 0.1 mL and 0.001 mL were pipetted and plated on separate blood agar plates (Columbia Agar w/5% sheep blood) and incubated in 37° C. for 24 hours. Colonies were counted and the resulting cfu/mL calculated. Controls were prepared the same as the treated dressings only without exposure to the NO-generating mixture.

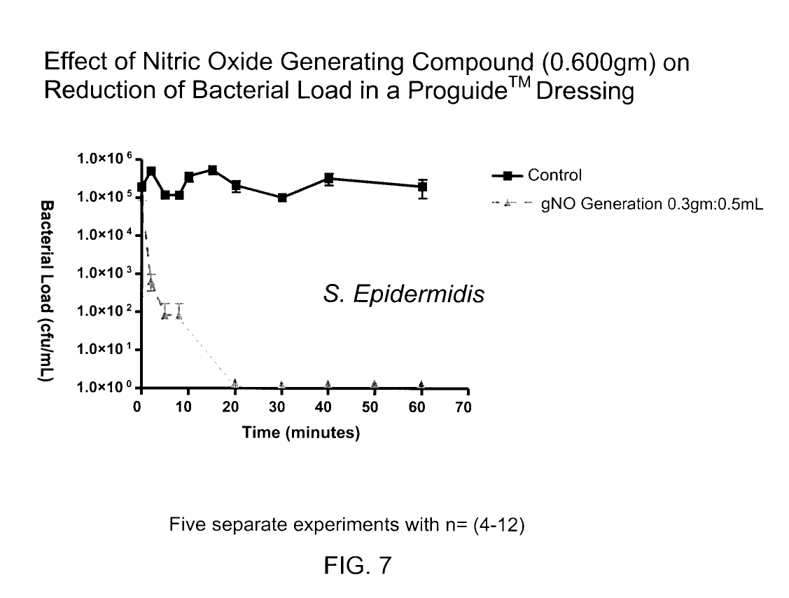

FIG. 7 shows the results of one set of experiments with the solid line plotted by square-shaped points representing the survival curve of the microorganisms in the control dressing and the dashed lines plotted by triangle-shaped points representing the survival curve of the dressings exposed to the NO-generating mixtures. Results were from five separate experiments with a total of 73 data points. These studies show that this combination of NO-generating mixture had greater than a 5 log₁₀ cfu/mL bactericidal effect on S. epidermidis within the dressing (Proguide, Smith & Nephew, England) within 20 minutes.

To characterize the NO, nitrogen dioxide (NO₂) and oxygen (O₂) production generated from various mixtures of NO-generating compounds a series of experiments were conducted. A standard 9 cm diameter Petri dish was modified to accommodate a sampling line (#5 Fr Feeding Tube, Mallinckrodt, USA) for the NO, NO₂, and O₂ analyzer (Aeronox, Pulmonox Medical Inc., Canada). Using a clean stainless steel spatula, three active ingredients were weighed and placed into the bottom of the petri dish and mixed thoroughly. The petri dish lid was then placed over the reactants. The gas analyzer sampling line was attached to the gas analyzer and readings were recorded when peak concentrations of NO, NO₂ and O₂ were attained.

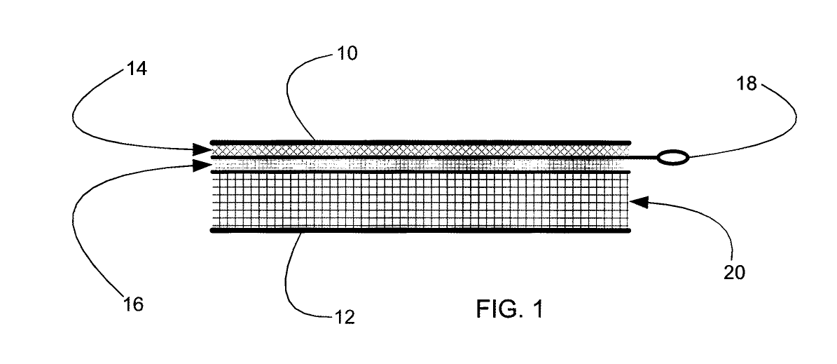

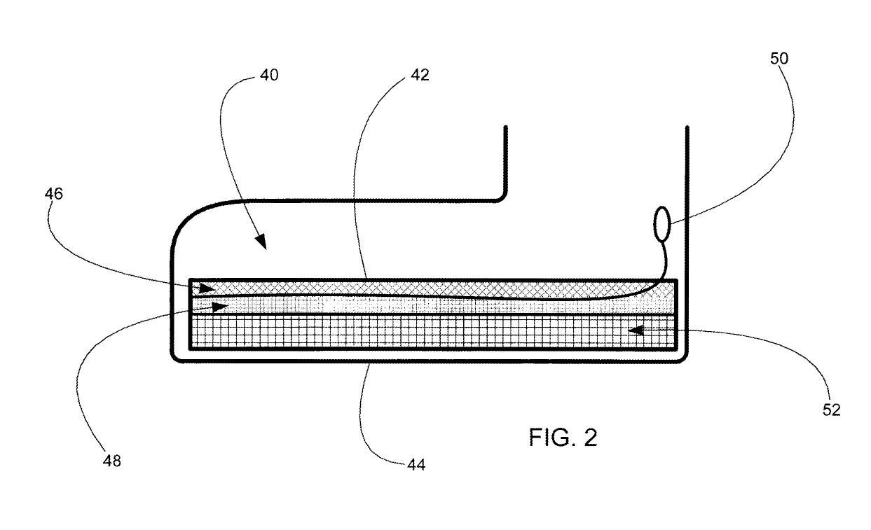

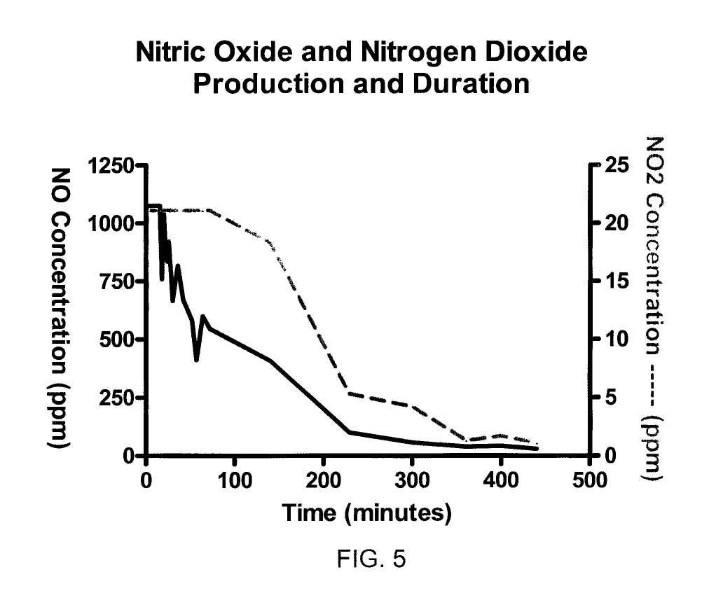

FIG. 5 shows the results from a series of experiments where three substances (SL 0.80 gm; SJ 1.21 gm; KY 1.32 gm) were mixed and analyzed. The solid blue line plotted represents the NO concentration on the left y-axis and the dashed red line plotted represents the NO₂ concentration on the right y-axis. The oxygen level was not plotted here. These data show that this ratio and amount of NO-generating material provided a peak of 800-1250 ppm NO for a over a 30 minute period. Experiments, such as these, were used to optimize the NO-generating compounds to identify the most effective combination of gases to provide a 100% bactericidal effect for the dressing studies. Over 10,000 ppm NO can be generated for over a half hour from as little a half a gram of NO-generating compounds. This finding makes it very feasible to design dressings that regulate specific concentrations and specific delivery durations for the embodiments described in FIGS. 1 and 2. For example, in FIG. 7 the three ingredients were 300 mg of each of two substances and 0.5 mL of another with the resulting NO/O₂ being a very effective combination to eradicate 10⁵ log₁₀ cfu/mL of S. epidermidis in 10 to 20 minutes from the Proguide Dressing.

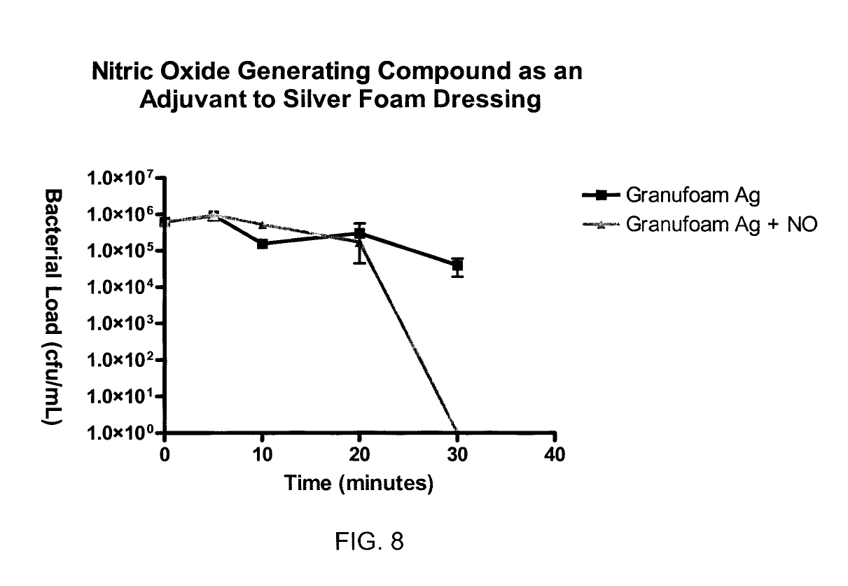

FIG. 8 shows the results of a study showing that NO-generating compounds act as an adjuvant to commercially available antimicrobial dressings. The solid blue line plotted by square-shaped points represents the survival curve of S. aureus for a silver foam dressing (V.A.C. Granufoam Silver, KCl, USA) and the solid red line triangle-shaped points plotted represents the survival curve of the silver foam dressing with the NO-generating compound. The data shows that the silver dressing purporting to be an anti-microbial dressing has only a one log₁₀ cfu/mL reduction in the S. aureus bacterial burden. Whereas, there is a complete bactericidal effect, during the same time period, for the same dressing used in conjunction with the NO-generating compound. These data demonstrate that an NO-generating dressing may accelerate the anti-microbial effect of currently commercially available anti-microbial dressings.

The foregoing description of embodiments of the present invention has been presented for the purpose of illustration and description. It is not intended to be exhaustive or to limit the invention to the particular forms disclosed. Obvious modifications and variations are possible in light of the above disclosure without departing from the spirit and scope of the present invention. The embodiments described were chosen to best illustrate the principles of the invention and practical applications thereof to enable one of ordinary skill in the art to utilize the invention in various embodiments and with various modifications as suited to the particular use contemplated. It is intended that the scope of the invention be defined by the claims appended hereto.

1-24. (canceled)

25. A wound dressing comprising a nitric oxide gas producing component and an oxygen releasing component.

26. A dressing according to claim 25 wherein the nitric oxide producing component comprises a discrete supply of sodium nitrite and a discrete supply of citric acid.

27. A dressing according to claim 26 wherein the dressing further comprises a discrete supply of water molecules.

28. A dressing according to claim 27 wherein the dressing comprises a plurality of layers, one layer comprising the discrete supply of sodium nitrite and citric acid, and a separate layer comprising the discrete supply of water molecules.

29. A dressing according to claim 27 wherein the supply of water molecules is selected from the group containing aqueous gels and/or sterile distilled water.

30. A dressing according to claim 27 wherein the discrete supply of water molecules is contained within a capsule and/or a frangible packet.

31. A dressing according to claim 29 wherein the supply of water molecules is controllably releasable.

32. A dressing according to claim 30 wherein the dressing comprises a device for applying a fracturing pressure to the capsule and/or the frangible packet.

33. A dressing according to claim 28 further comprising a separate layer containing an oxygen releasing component.

34. A dressing according to claim 33 wherein the oxygen releasing component produces oxygen to a concentration of 18% or greater.

35. A wound dressing comprising:
an upper surface layer comprising a gas-impermeable and moisture-impermeable membrane;
a bottom surface layer barrier comprising a gas-permeable membrane;
an oxygen releasing component;
a discrete supply of a chemical mixture configured for producing a discrete amount of NO gas when said discrete supply of the chemical mixture is commingled with a supply of water molecules; and
a discrete supply of water molecules.

36. A dressing according to claim 35 wherein the chemical mixture comprises a discrete supply of sodium nitrite and a discrete supply of citric acid.

37. A dressing according to claim 35 wherein the chemical mixture is contained within one layer and the supply of water molecules is contained within a separate layer.

38. A dressing according to claim 35 wherein the supply of water molecules is selected from the group containing aqueous gels and/or sterile distilled water.

39. A dressing according to claim 35 wherein the supply of water molecules is contained within a capsule and/or a frangible packet.

40. An apparatus comprising a nitric oxide gas producing component wherein the component comprises a discrete supply of sodium nitrite, and a discrete supply of citric acid, and a discrete supply of an oxygen-releasing component, the apparatus being selected from inner-soles, socks, or other type of footwear.

41. An apparatus according to claim 40 further comprising a discrete supply of water molecules, the water molecules being contained within a frangible capsule and/or a packet.

42. An apparatus according to claim 40, the apparatus comprising:
a first layer comprising a discrete supply of sodium nitrite and a discrete supply of citric acid;
a second layer comprising a discrete supply of water molecules, the water molecules being contained within a frangible capsule and/or packet; and
a discrete supply of an oxygen-releasing component.

43. An apparatus according to claim 41 wherein the apparatus comprises a pull-tab device attached to said frangible capsule or packet which, when pulled, fractures said capsule or packet.

44. A method for providing an anti-septic treatment to a portion of a subject's body surface, said method comprising the steps of:
overlaying a selected portion of the subject's body surface with a wound dressing assembly according to claim 25; and
causing NO gas to be produced.

45. A method according to claim 44, wherein the concentration of gaseous NO is from about 5,000 to 22,000 ppm.
